(19) United States
(12) Patent Application Publication
(10) Pub. No.: US 2016/0051543 A1
(43) Pub. Date: Feb. 25, 2016

(54) METHODS OF TREATMENT OF CARDIAC ARRHYTHMIAS USING VANOXERINE AND MODIFICATION OF DIET

(71) Applicant: CHANRX CORPORATION, Cleveland, OH (US)

(72) Inventor: Arthur M. Brown, Brecksville, OH (US)

(21) Appl. No.: 14/784,860

(22) PCT Filed: Apr. 25, 2014
(86) PCT No.: PCT/US14/35564

§ 371 Date: Oct. 15, 2015

Related U.S. Application Data
(60) Provisional application No. 61/816,214, filed on Apr. 26, 2013.
(60) Provisional application No. 61/904,813, filed on Nov. 15, 2013.
(60) Provisional application No. 61/915,792, filed on Dec. 13, 2013.

(51) Int. Cl. A61K 31/495; A23L 1/30; G01N 33/94

(52) U.S. Cl. CPC A61K 31/495; G01N 33/9453; A23L 1/3006; G01N 2800/326; G01N 2800/52; A23V 2002/00

(57) ABSTRACT

Disclosed embodiments are related to methods of treatment of cardiac arrhythmias comprising administration of vanoxerine (GBR 12909) in connection with food of a predetermined fat content to modulate the plasma level concentrations of vanoxerine in a patient.

### FIELD OF THE INVENTION

Presently disclosed embodiments are related to methods administration of vanoxerine and food for terminating acute episodes of cardiac arrhythmia, preventing the same, restoring normal sinus rhythm, preventing re-occurrence of cardiac arrhythmia, and maintaining normal sinus rhythm in mammals. Presently disclosed embodiments particularly relate to methods for dosing and treatment methodologies utilizing vanoxerine administered concurrently with a modified diet to increase efficacy of the vanoxerine.

### BACKGROUND

Vanoxerine (1-[2-[bis(4-fluorophenyl)methoxy]ethyl]-4-(3-phenylpropyl)piperazine), its manufacture and/or certain pharmaceutical uses thereof are described in U.S. Pat. No. 4,202,896, U.S. Pat. No. 4,476,129, U.S. Pat. No. 4,874,765, U.S. Pat. No. 6,743,797 and U.S. Pat. No. 7,700,600, as well as European Patent EP 243,903 and PCT International Application WO 91/01732, each of which is incorporated herein by reference in its entirety.

Vanoxerine has been used for treating cocaine addiction, acute effects of cocaine, and cocaine cravings in mammals, as well as dopamine agonists for the treatment of Parkinsonism, acromegaly, hyperprolactinemia and diseases arising from a hypofunction of the dopaminergic system. (See U.S. Pat. No. 4,202,896 and WO 91/01732). Vanoxerine has also been used for treating and preventing cardiac arrhythmia in mammals. (See U.S. Pat. No. 6,743,797 and U.S. Pat. No. 7,700,600).

Atrial flutter and/or atrial fibrillation (AF) are the most commonly sustained cardiac arrhythmias in clinical practice, and are likely to increase in prevalence with the aging of the population. Currently, AF affects more than 1 million Americans annually, represents over 5% of all admissions for cardiovascular diseases and causes more than 80,000 strokes each year in the United States. In the US alone, AF currently afflicts more than 2.3 million people. By 2050, it is expected that there will be more than 12 million individuals afflicted with AF. While AF is rarely a lethal arrhythmia, it is responsible for substantial morbidity and can lead to complications such as the development of congestive heart failure or thromboembolism. Currently available Class I and Class III anti-arrhythmic drugs reduce the rate of re-occurrence of AF, but are of limited use because of a variety of potentially adverse effects, including ventricular proarrhythmia. Because current therapy is inadequate and fraught with side effects, there is a clear need to develop new therapeutic approaches.

Current first line pharmacological therapy options for AF include drugs for rate control. Despite results from several studies suggesting that rate control is equivalent to rhythm control, many clinicians believe that patients are likely to have better functional status when in sinus rhythm. Further, being in AF may introduce long-term mortality risk, where achievement of rhythm control may improve mortality.

Ventricular fibrillation (VF) is the most common cause associated with acute myocardial infarction, ischemic coronary artery disease and congestive heart failure. As with AF, current therapy is inadequate and there is a need to develop new therapeutic approaches.

Although various anti-arrhythmic agents are now available on the market, those having both satisfactory efficacy and a high margin of safety have not been obtained. For example, anti-arrhythmic agents of Class I, according to the classification scheme of Vaughan-Williams (“Classification of antiarrhythmic drugs,” Cardiac Arrhythmias, edited by: E. Sandoe, E. Flensted-Jensen, K. Olesen; Sweden, Astra, Sodertalje, pp 449-472 (1981)), which cause a selective inhibition of the maximum velocity of the upstroke of the action potential (Vₘₐₓ) are inadequate for preventing ventricular fibrillation because they shorten the wave length of the cardiac action potential, thereby favoring re-entry. In addition, these agents have problems regarding safety, i.e. they cause a depression of myocardial contractility and have a tendency to induce arrhythmias due to an inhibition of impulse conduction. The CAST (coronary artery suppression trial) study was terminated while in progress because the Class I antagonists had a higher mortality than placebo controls. β-adrenergenic receptor blockers and calcium channel (I_{Ca}) antagonists, which belong to Class II and Class IV, respectively, have a defect in that their effects are either limited to a certain type of arrhythmia or are contraindicated because of their cardiac depressant properties in certain patients with cardiovascular disease. Their safety, however, is higher than that of the anti-arrhythmic agents of Class I.

Prior studies have been performed using single dose administration of flecainide or propafenone (Class I drugs) in terminating atrial fibrillation. Particular studies investigated the ability to provide patients with a known dose of one of the two drugs so as to self-medicate should cardiac arrhythmia occur. P. Alboni, et al., “Outpatient Treatment of Recent-Onset Atrial Fibrillation with the ‘Pill-in-the-Pocket’ Approach,” NEJM 351; 23 (2004); L. Zhou, et al., “‘A Pill in the Pocket’ Approach for Recent Onset Atrial Fibrillation in a Selected Patient Group,” Proceedings of UCLA Healthcare 15 (2011). However, the use of flecainide and propafenone has been criticized as including candidates having structural heart disease and thus providing patients likely to have risk factors for stroke who should have received antithrombotic therapy, instead of the flecainide or propafenone. NEJM 352:11 (Letters to the Editor) (Mar. 17, 2005). Similarly, the use of warfarin concomitantly with propafenone was criticized.

Anti-arrhythmic agents of Class III are drugs that cause a selective prolongation of the action potential duration (APD) without a significant depression of the maximum upstroke velocity (Vₘₐₓ). They therefore lengthen the save length of the cardiac action potential increasing refractories, thereby antagonizing re-entry. Available drugs in this class are limited in number. Examples such as sotalol and amiodarone have been shown to possess interesting Class III properties (Singh B. N., Vaughan Williams E. M., “A Third Class of Anti-Arrhythmic Action: Effects on Atrial and Ventricular Intracellular Potentials and other Pharmacological Actions on Cardiac Muscle of MJ 1999 and AH 3747,” (Br. J. Pharmacol 39:675-689 (1970), and Singh B. N., Vaughan Williams E. M., “The Effect of Amiodarone, a New Anti-Anginal Drug, on Cardiac Muscle,” Br. J. Pharmacol 39:657-667 (1970)), but these are not selective Class III agents. Sotalol also possesses Class II (β-adrenergic blocking) effects which may cause cardiac depression and is contraindicated in certain susceptible patients.

Amiodarone also is not a selective Class III antiarrhythmic agent because it possesses multiple electrophysiological actions and is severely limited by side effects. (Nademanee, K., “The Amiodarone Odyssey,” J. Am. Coll. Cardiol. 20:1063-1065 (1992)). Drugs of this class are expected to be effective in preventing ventricular fibrillation. Selective Class III agents, by definition, are not considered to cause myocardial depression or an induction of arrhythmias due to inhibition of conduction of the action potential as seen with Class I antiarrhythmic agents.

Class III agents increase myocardial refractoriness via a prolongation of cardiac action potential duration (APD). Theoretically, prolongation of the cardiac action potential can be achieved by enhancing inward currents (i.e. Na+ or Ca²+ currents; hereinafter I_{Na} and I_{Ca}, respectively) or by reducing outward repolarizing potassium K+ currents. The delayed rectifier (I_{K}) K+ current is the main outward current involved in the overall repolarization process during the action potential plateau, whereas the transient outward (Iₜₒ) and inward rectifier (I_{KI}) K+ currents are responsible for the rapid initial and terminal phases of repolarization, respectively.

Cellular electrophysiologic studies have demonstrated that I_{K} consists of two pharmacologically and kinetically distinct K+ current subtypes, I_{K} (rapidly activating and deactivating) and I_{Ks} (slowly activating and deactivating). (Sanguinetti and Jurkiewicz, “Two Components of Cardiac Delayed Rectifier K+ Current. Differential Sensitivity to Block by Class III Anti-Arrhythmic Agents,” J Gen Physiol 96:195-215 (1990)). I_{Kr} is also the product of the human ether-a-go-go gene (hERG). Expression of hERG cDNA in cell lines leads to production of the hERG current which is almost identical to I_{Kr} (Curran et al., “A Molecular Basis for Cardiac Arrhythmia: hERG Mutations Cause Long QT Syndrome,” Cell 80(5):795-803 (1995)).

Class III anti-arrhythmic agents currently in development, including d-sotalol, dofetilide (UK-68,798), almokalant (H234/09), E-4031 and methanesulfonamide-N-[1′-6-cyano-1,2,3,4-tetrahydro-2-naphthalenyl)-3,4-dihydro-4-hydroxyspiro[2H-1-benzopyran-2,4′-piperidin]-6yl], (+)-, monochloride (MK-499) predominantly, if not exclusively, block I_{Kr}. Although amiodarone is a blocker of I_{Ks} (Balser J. R. Bennett, P. B., Hondeghem, L. M. and Roden, D. M. “Suppression of time-dependent outward current in guinea pig ventricular myocytes: Actions of quinidine and amiodarone,” Circ. Res. 69:519-529 (1991)), it also blocks I_{Na} and I_{Ca}, effects thyroid function, as a nonspecific adrenergic blocker, acts as an inhibitor of the enzyme phospholipase, and causes pulmonary fibrosis (Nademanee, K., “The Amiodarone Odessey.” J. Am. Coll. Cardiol. 20:1063-1065 (1992)).

Reentrant excitation (reentry) has been shown to be a prominent mechanism underlying supraventricular arrhythmias in man. Reentrant excitation requires a critical balance between slow conduction velocity and sufficiently brief refractory periods to allow for the initiation and maintenance of multiple reentry circuits to coexist simultaneously and sustain AF. Increasing myocardial refractoriness, by prolonging APD, prevents and/or terminates reentrant arrhythmias. Most selective Class III antiarrhythmic agents currently in development, such as d-sotalol and dofetilide predominantly, if not exclusively, block I_{KR}, the rapidly activating component of I_{K} found both in atria and ventricle in man.

Since these I_{Kr} blockers increase APD and refractoriness both in atria and ventricle without affecting conduction per se, theoretically they represent potential useful agents for the treatment of arrhythmias like AF and VF. These agents have a liability in that they have an enhanced risk of proarrhythmia at slow heart rates. For example, torsade de pointes, a specific type of polymorphic ventricular tachycardia which is commonly associated with excessive prolongation of the electrocardiographic QT interval, hence termed “acquired long QT syndrome,” has been observed when these compounds are utilized (Roden, D. M., “Current Status of Class III Antiarrhythmic Drug Therapy,” Am J. Cardiol, 72:44B-49B (1993)). The exaggerated effect at slow heart rates has been termed “reverse frequency-dependence” and is in contrast to frequency-independent or frequency-dependent actions. (Hondeghem, L. M., “Development of Class III Antiarrhythmic Agents,” J. Cardiovasc. Cardiol. 20 (Suppl. 2):S17-S22). The pro-arrhythmic tendency led to suspension of the SWORD trial when d-sotalol had a higher mortality than placebo controls.

The slowly activating component of the delayed rectifier (I_{Ks}) potentially overcomes some of the limitations of I_{Kr} blockers associated with ventricular arrhythmias. Because of its slow activation kinetics, however, the role of I_{Ks} in atrial repolarization may be limited due to the relatively short APD of the atrium. Consequently, although I_{Ks} blockers may provide distinct advantage in the case of ventricular arrhythmias, their ability to affect supraventricular tachyarrhythmias (SVT) is considered to be minimal.

Another major defect or limitation of most currently available Class III anti-arrhythmic agents is that their effect increases or becomes more manifest at or during bradycardia or slow heart rates, and this contributes to their potential for proarrhythmia. On the other hand, during tachycardia or the conditions for which these agents or drugs are intended and most needed, they lose most of their effect. This loss or diminishment of effect at fast heart rates has been termed “reverse use-dependence” (Hondeghem and Snyders, “Class III antiarrhythmic agents have a lot of potential but a long way to go: Reduced Effectiveness and Dangers of Reverse use Dependence,” Circulation, 81:686-690 (1990); Sadanaga et al., “Clinical Evaluation of the Use-Dependent QRS Prolongation and the Reverse Use-Dependent QT Prolongation of Class III Anti-Arrhythmic Agents and Their Value in Predicting Efficacy,” Amer. Heart Journal 126:114-121 (1993)), or “reverse rate-dependence” (Bretano, “Rate dependence of class III actions in the heart,” Fundam. Clin. Pharmacol. 7:51-59 (1993); Jurkiewicz and Sanguinetti, “Rate-Dependent Prolongation of Cardiac Action Potentials by a Methanesulfonanilide Class III Anti-Arrhythmic Agent: Specific Block of Rapidly Activating Delayed Rectifier K+ current by Dofetilide,” Circ. Res. 72:75-83 (1993)). Thus, an agent that has a use-dependent or rate-dependent profile, opposite that possessed by most current class III anti-arrhythmic agents, should provide not only improved safety but also enhanced efficacy.

Vanoxerine has been indicated for treatment of cardiac arrhythmias. Indeed, certain studies have looked at the safety profile of vanoxerine and stated that no side-effects should be expected with a daily repetitive dose of 50 mg of vanoxerine. (U. Sogaard, et. al., “A Tolerance Study of Single and Multiple Dosing of the Selective Dopamine Uptake Inhibitor GBR 12909 in Healthy Subjects,” International Clinical Psychopharmacology, 5:237-251 (1990)). However, Sogaard, et. al. also found that upon administration of higher doses of vanoxerine, some effects were seen with regard to concentration difficulties, increase systolic blood pressure, asthenia, and a feeling of drug influence, among other effects. Sogaard, et. al. also recognized that there were unexpected fluctuations in serum concentrations with regard to these healthy patients. While they did not determine the reasoning, control of such fluctuations may be important to treatment of patients.

Further studies have looked at the ability of food to lower the first-pass metabolism of lipophilic basic drugs, such as vanoxerine. (S. H. Ingwersen, et. al., “Food Intake Increases the Relative Oral Bioavailability of Vanoxerine,” Br. J. Clin. Pharmac; 35:308-130 (1993)). However, no methods have been utilized or identified for treatment of cardiac arrhythmias in conjunction with the modulating effects of food intake.

Accordingly, new methods are required to improve treatment and efficacy of vanoxerine in the treatment of patients suffering from cardiac arrhythmia by utilizing modification of food intake and/or diet to ameliorate treatment with vanoxerine.

### SUMMARY

Embodiments of the present disclosure relate to methods of treatment of cardiac arrhythmias comprising administration of vanoxerine (GBR 12909) in connection with food of a predetermined fat content to modulate the physiological concentrations of vanoxerine in a patient.

Further embodiments of the present disclosure relate to methods for treating cardiac arrhythmias comprising: determining a physiological concentration of vanoxerine effective for treating cardiac arrhythmias; determining an appropriate vanoxerine dose to be administered with a meal; instructing a patient to consume a pre-determined meal; and thereafter administering the determined dose of vanoxerine.

Further embodiments of the present disclosure relate to methods for treating cardiac arrhythmias comprising: instructing a patient to consume a high fat meal and concurrently administering a first dose of vanoxerine; measuring the physiological concentration of vanoxerine; modifying the meal to increase or decrease the fat content of the meal based on the plasma level concentration; and instructing patient to modify the fat content of a meal taken with a subsequently administered dose of vanoxerine.

Further embodiments of the present disclosure relate to methods for treating cardiac arrhythmias comprising: administering a first dose of vanoxerine to a patient; measuring the physiological concentration of vanoxerine and/or one or more metabolites of vanoxerine in said patient; comparing the physiological level concentration to a pre-determined physiological concentration; instructing the patient to consume a meal having a pre-determined fat content concomitantly with a subsequent administration of vanoxerine.

Other aspects of the present invention comprise methods for terminating acute episodes of cardiac arrhythmia, such as atrial fibrillation or ventricular fibrillation, in a mammal, such as a human, by administering a first dose of vanoxerine; measuring the plasma level concentration of vanoxerine and/or one or more metabolites of vanoxerine; comparing the plasma level concentration to a pre-determined plasma level; and instructing the patient to consume a meal having a pre-determined fat content concomitantly with a subsequent administration of vanoxerine.

Another aspect of the present invention is directed to a method for restoring normal sinus rhythm in a mammal, such as a human patient, exhibiting cardiac arrhythmia by administering a first dose of vanoxerine to said patient; measuring the plasma level concentration of vanoxerine and/or one or more metabolites of vanoxerine; comparing the plasma level concentration to a pre-determined plasma level; instructing the patient to consume a meal having a pre-determined fat content concomitantly with a subsequent administration of vanoxerine so as to modify the bioavailability of the administered vanoxerine.

Another aspect of the present invention is directed to a method for maintaining normal sinus rhythm in a mammal, such as a human patient, by administering a first dose of vanoxerine to said patient; measuring the physiological concentration of vanoxerine and/or one or more metabolites of vanoxerine; comparing the physiological concentration to a pre-determined plasma level; instructing the patient to consume a meal having a pre-determined fat content concomitantly with a subsequent administration of vanoxerine so as to modify the bioavailability of the administered vanoxerine.

Another aspect of the present disclosure is directed to a method for preventing a re-occurrence of an episode of cardiac arrhythmia in a mammal, such as a human, by administering a first dose of vanoxerine; measuring the physiological concentration of vanoxerine and/or one or more metabolites of vanoxerine; comparing the physiological concentration to a pre-determined physiological level; instructing the patient to consume a meal having a pre-determined fat content concomitantly with a subsequent administration of vanoxerine so as to modify the bioavailability of the administered vanoxerine.

A further embodiment is a method for modulating plasma level concentrations in a patient being treated for cardiac arrhythmia comprising: administering a first dose of vanoxerine; measuring the plasma level of vanoxerine; calculating an effective dose of vanoxerine to be taken after a high-fat meal; and instructing patient to consume a high-fat meal and immediately thereafter consuming an effective dose of vanoxerine.

A further embodiment is a method of administration of vanoxerine to a patient comprising administering a first dose of vanoxerine to a patient under fasting conditions and administering a second dose of vanoxerine to said same patient about 1-2 hours after said first administration, wherein said second dose is taken concurrently with a high-fat meal.

Additional aspects of the present disclosure are directed to methods for modulation of Cₘₐₓ (maximum peak concentration) and tₘₐₓ (the time after administration when the drug reaches maximum plasma concentration) with regard to a particular patient, wherein a first effective dose of a drug comprising vanoxerine is administered; the physiological concentration is measured subsequent to administration; the Cₘₐₓ and tₘₐₓ are determined for the patient; a plan for diet modification is determined based on the physiological concentration (from plasma, blood, or other tissues); wherein the patient is instructed to consume a modified meal concomitantly with administration of a second effective dose of vanoxerine.

A method for administering vanoxerine for treatment of cardiac arrhythmia comprising: administering a first dose of vanoxerine to a patient; determining the bioavailability of the patient by measuring the physiological concentration of vanoxerine; calculating an effective dose of vanoxerine to be administered with a meal of a pre-determined fat content to be taken concurrently to modify the physiological concentration; and administering the effective dose of vanoxerine with the pre-determined meal.

A method for achieving a pre-determined plasma level comprising: administering a first dose of vanoxerine concurrently with a high-fat meal; measuring the physiological concentration of vanoxerine; comparing the physiological concentration to the pre-determined physiological concentration; modifying a further dose of vanoxerine to be given concurrently with a high-fat meal; and administering the second dosage of vanoxerine in conjunction with the high-fat meal.

A method of minimizing variability of physiological concentrations for treatment of cardiac arrhythmia with vanoxerine comprising: determining a target physiological concentration; administering a first dose of a drug comprising vanoxerine to a patient; measuring the physiological concentration of vanoxerine in said patient; and instructing patient to consumer a high-fat meal concurrently with a further dose of vanoxerine.

Administering steps in any of the foregoing methods may comprise administration by a caregiver, a medical professional, or self-administered by a patient.

### DETAILED DESCRIPTION OF PREFERRED EMBODIMENTS

All references cited herein are hereby incorporated by reference in their entirety.

As used herein, the term “about” is intended to encompass a range of values ±10% of the specified value(s). For example, the phrase “about 20” is intended to encompass ±10% of 20, i.e. from 18 to 22, inclusive.

As used herein, the term “vanoxerine” refers to vanoxerine and pharmaceutically acceptable salts thereof.

As used herein, the term “pharmaceutically acceptable” refers to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable benefit/risk ratio.

As used herein, the term “subject” refers to a warm blooded animal such as a mammal, preferably a human or a human child, which is afflicted with, or has the potential to be afflicted with one or more diseases and conditions described herein.

As used herein, “therapeutically effective amount” refers to an amount which is effective in reducing, eliminating, treating, preventing or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment.

As used herein, “unit dose” means a single dose which is capable of being administered to a subject, and which can be readily handled and packaged, remaining as a physically and chemically stable unit dose comprising either vanoxerine or a pharmaceutically acceptable composition comprising vanoxerine.

As used herein, “administering” or “administer” refers to the actions of a medical professional or caregiver, or alternatively self-administration by the patient.

The term “steady state” means wherein the overall intake of a drug is fairly in dynamic equilibrium with its elimination.

As used herein, a “pre-determined” plasma level or other physiological tissue or fluid and refers to a concentration of vanoxerine at a given time point. Typically, a pre-determined level will be compared to a measured level, and the time point for the measured level will be the same as the time point for the pre-determined level. In considering a pre-determined level with regard to steady state concentrations, or those taken over a period of hours, the pre-determined level is referring to the mean concentration taken from the area under the curve (AUC), as the drug increases and decreases in concentration in the body with regard to the addition of a drug pursuant to intake and the elimination of the drug via bodily mechanisms.

Cardiac arrhythmias include atrial, junctional, and ventricular arrhythmias, heart blocks, sudden arrhythmic death syndrome, and include bradycardias, tachycardias, re-entrant, and fibrillations. These conditions, including the following specific conditions: atrial flutter, atrial fibrillation, multifocal atrial tachycardia, premature atrial contractions, wandering atrial pacemaker, supraventricular tachycardia, AV nodal reentrant tachycardia, junctional rhythm, junctional tachycardia, premature junctional contraction, premature ventricular contractions, ventricular bigeminy, accelerated idioventricular rhythm, monomorphic ventricular tachycardia, polymorphic ventricular tachycardia, and ventricular fibrillation, and combinations thereof are all capable of severe morbidity and death if left untreated. Methods and compositions described herein are suitable for the treatment of these and other cardiac arrhythmias.

Consumption of food directly before the administration of vanoxerine has certain impacts on plasma concentrations. One study identified the potential for food effects on patients. (See S. H. Ingwersen, et al.). Ingwersen defined food consumption based on one of three categories: fasting (no food intake for eight hours), low-fat meal (about 3 grams of fat), or a high-fat meal (about 73 g fat). While the Cₘₐₓ is not significantly different between fasting and low-fat diets, the high-fat diet has a significantly increased Cₘₐₓ. Interestingly Ingwersen, et al. found that the area under the curve (AUC) varied dramatically based upon three food options, fasting, low fat, and high fat. The studies identified that fasting has the lowest AUC, low-fat a significant increase above fasting, and high fat having more than twice the AUC total of the low-fat diet. Finally, with regard to tₘₐₓ, fasting has the fastest time to tₘₐₓ, with low-fat diet slightly slower, and the high-fat diet taking nearly two and a half hours until tₘₐₓ.

Accordingly, methods may be utilized to manipulate and modify the bioavailability of vanoxerine for a patient using food in the treatment of cardiac arrhythmias. The use of a high-fat diet promotes Cₘₐₓ as well as the AUC with regard to vanoxerine. However, a high-fat diet also extends the time from administration for tₘₐₓ. Where a high AUC or Cₘₐₓ is desired, a method of consumption of a high-fat diet immediately preceding the administration of the vanoxerine provides for an improved bioavailability.

However, in circumstances where a patient has previously taken vanoxerine, or it is otherwise determined that the patient does not have concerns with bioavailability, it may be beneficial to utilize a fasting diet or a low-fat diet to improve tₘₐₓ or the pharmacokinetic profile of the drug in a patient. In particular situations, a faster tₘₐₓ may provide a more immediate impact on restoring normal sinus rhythm or arresting an episode of cardiac arrhythmia where a later acting dose would be less beneficial. Furthermore, fasting provides the fastest elimination of vanoxerine from the body, which may be necessary or desired in some circumstances.

While a high-fat meal does not necessarily mean that efficacious physiological concentrations (including blood, plasma, and other tissues) are reached later than with a fasting or a low-fat diet, the low fat and fasting diets have a profile that essentially spikes at or around an hour post administration, and then the plasma concentration dropping rapidly. With the high-fat diet, the concentration remains elevated for four to six hours and then gradually tapering, thus resulting in a much larger AUC than with the other diets. In some scenarios, each of the profiles may be advantageous.

Accordingly, in embodiments where it is advantageous to maintain a stable, elevated physiological concentration, a method includes instructing a patient to consume a high-fat diet (about 70 g fat), about 1 to 2 hours to about 1 minute before administration of a dose of vanoxerine; instructing said patient to consume an additional high-fat diet about 6-8 hours later and administering at least a second dose of vanoxerine. In some embodiments, the vanoxerine can be administered concurrently with a high-fat meal.

Where it would be advantageous to maintain such elevated levels, a small dose of vanoxerine may be utilized and the dose, given twice or three times daily would be taken with a high-fat meal, either immediately preceding the administration or concurrently. Such high-fat foods might include animal products—including meat, dairy, or the like. Some particular examples include whole milk, egg, butter, red meat, oils. A high-fat diet means consumption of about 20 to about 100 g fat concurrently with administration of the vanoxerine. More particularly, a high-fat food, meal, or diet means about 25 to about 75 g of fat, or about 50 to about 75 g of fat. A low fat diet means about 1 to about 15 g of fat, or about 1 to about 10 g of fat, or about 1 to about 5 g of fat. The high-fat food, or high-fat meal, or high-fat diet, may mean one or more foods taken immediately before administration of vanoxerine, where the food is consumed less than an hour before administration, or taken concurrently with the vanoxerine dose. A single food or multiple foods may sufficiently provide the necessary fat content.

Conversely, fasting should include no food intake for about 4 hours, and preferably more than 6 or 8 hours. In some circumstances no food in about 2 hours may be sufficient to see the food effects of fasting.

Vanoxerine has a relatively long plasma half-life of about 22 hours, and further tests suggest that repetitive dosing in dogs provides a half-life is considerably longer at about 66 hours. Further studies have suggested that the half-life may extend up to 125 hours in some cases. These studies have reported that in some cases steady state is achieved within 3 days of oral dosing. Indeed, tests on recovery of administration of radioactivity labeled vanoxerine in rats were incomplete. This, coupled with the observed biliary excretion, suggests enterohepatic circulation may be occurring. This provides for an opportunity to achieve steady state plasma levels for restoration or maintenance of normal sinus rhythm in mammals. Through the use of pre-determined fat content meals taken with vanoxerine, improvements can be made with regard to reaching steady state quickly and efficiently, and variability of concentrations due to pharmacokinetic metabolism can be minimized.

Efficacious target plasma level concentrations, taken at a time point of 1 hour post administration are about 5 to about 1000 ng/ml. In alternative embodiments, physiological concentrations, as measured in the plasma at a time of 1 hour post administration are about 20 to about 400 ng/ml, or about 20 to about 200 ng/ml, or about 25 to about 150 ng/ml or about 40 to about 125 ng/ml, or about 60 to about 100 ng/ml. In measuring plasma levels for confirmation of half-life and/or steady state plasma levels, it may be necessary to take additional plasma level measurements at further time points, such as 2, 4, 6, 8, 12, 24, 36, 48, 72, hours, and other times as appropriate. In some cases, it may be advantageous to test plasma levels every 24, 48, 72, or 96 hours, or to test plasma levels prior to or subsequent to a further administration of vanoxerine.

To reach these concentrations, in some embodiments, a dosage of 1 mg to 1000 mg vanoxerine per unit dose is appropriate. Other embodiments may utilize a dosage of about 25 mg to 500 mg, or about 25 to 400 mg, or about 50 mg to about 400 mg, or about 200 to about 400 mg. Preferred embodiments include administration of vanoxerine in about 25, 50, 75, 100, 125, 150, 200, 300, and 400 mg doses for daily dosing or a loading period and for maintenance amounts for treatment of chronic cardiac arrhythmia.

Studies have also identified that human subjects have variability with regard to the metabolism of vanoxerine. In particular, studies identified that patients typically fall into one of two groups, a fast or a slow metabolizer. However, even within these groupings, there appears to still be significant variability between patients. Accordingly, there exists, even within the groupings, a continuum that provides that some people are faster or slower metabolizers even within the groups. Accordingly, in one embodiment, a method of treatment may be further personalized by recognizing that an individual patient metabolizes vanoxerine differently than another patient. Accordingly, whether a patient would benefit from fasting, low-fat diet, or a high-fat diet is fully dependent on the individual. Yet, information about the patient's general profile, i.e. whether they are a slower or faster metabolizer is still valuable to understanding their entire profile and variability, as those in slower groups versus higher metabolism groups tend to have Cₘₐₓ concentrations or an AUC that are 10 fold or more different than another patient.

In other embodiments, it is advantageous to provide for a certain dose, or a maximum dose at a given time point after administration of the vanoxerine to safely and effectively treat the cardiac arrhythmia. Accordingly, modification of Cₘₐₓ and tₘₐₓ is appropriate to maintain consistent Cₘₐₓ plasma level concentrations for a particular patient. Cₘₐₓ levels are preferably about 5 to about 1000 ng/ml. In alternative embodiments, plasma level concentrations at 1 hour post administration are about 10 to about 400 ng/ml, or about 20 to about 200 ng/ml, or about 20 to about 150 ng/ml, or about 25 to about 125 ng/ml or about 40 to about 100 ng/ml, and about 60 to about 100 ng/ml. Conversely tₘₐₓ is appropriately reached at about 1 hour post administration. In other embodiments, tₘₐₓ is appropriately reached at about 30 minutes, or about 90 minutes, or about 120 minutes, or about 240 minutes post administration. These maximum values vary widely by patient and modification of the dose, of the dosing schedule, of diet, and of other concomitant medications may be utilized to reach a predetermined therapeutic level.

Accordingly, a further embodiment comprises a method of administration of vanoxerine comprising a first administration of vanoxerine to a patient; measuring of the plasma level of vanoxerine in said patient; determining the metabolic profile of the patient based on the plasma levels and the vanoxerine administered to said patient; determining an appropriate modification of diet so as to modify the bioavailability of vanoxerine; instructing the patient to fast, consume a low-fat meal, or a high-fat meal based on the modification necessary, and administering the vanoxerine to the patient concurrently with the meal.

Further steps may be modified with the methods described herein. It may be appropriate to first determine the patient's metabolic profile with regard to vanoxerine. It may also be advantageous to determine the patient's bioavailability for vanoxerine. Then, based on the known profile of the patient, a vanoxerine dose may be determined by comparing the patients profile to a known profile and modifying diet and dose of vanoxerine to improve the efficacy and precision of the treatment. Further, after a first administration of vanoxerine, further plasma levels may be determined and additional modifications to the diet and dose may be made to further improve the efficacy of the vanoxerine treatment. In certain situations further plasma levels may be taken to monitor and/or modify the administration of vanoxerine.

Additionally, maintenance of elevated plasma levels over the course of at least 4 hours may be achieved. Elevated plasma levels may be desired over the course of about 4 to about 24 hours, or alternatively, over the course of about 12 hours to about 72 hours. Further embodiments provide for benefits for elevated plasma levels over the course of days and/or weeks. Accordingly, methods utilizing high-fat food intake immediately preceding or concurrently with vanoxerine may provide for a slow-release or delayed release type product without the need for difficult formulation. Accordingly, an embodiment comprises administration of one or more doses of vanoxerine taken concomitantly with a high-fat diet.

Further embodiments include a method of administration of a first dose of vanoxerine on an empty stomach (no food for about 2 to about 6 hours) followed up by a second dose of vanoxerine given about 2 hours after the first dose was administered, wherein said second dose is given concurrently with a high-fat meal. Therefore, the administration provides for a fast Tₘₐₓ followed by a dose for increasing the AUC and Cₘₐₓ.

Physiological levels can be determined by a number of substances such as blood, plasma, or other tissue from the patient. Other methods may also be used to measure the protein binding to determine the free concentration of vanoxerine in the body as is known to one of skill in the art.

Suitable methods for treatment of cardiac arrhythmias include various dosing schedules which may be administered by any technique capable of introducing a pharmaceutically active agent to the desired site of action, including, but not limited to, buccal, sublingual, nasal, oral, topical, rectal and parenteral administration. Dosing may include single daily doses, multiple daily doses, single bolus doses, slow infusion injectables lasting more than one day, extended release doses, IV or continuous dosing through implants or controlled release mechanisms, and combinations thereof. These dosing regimens in accordance with the method allow for the administration of the vanoxerine in an appropriate amount to provide an efficacious level of the compound in the blood stream or in other target tissues. Delivery of the compound may also be through the use of controlled release formulations in subcutaneous implants or transdermal patches.

For oral administration, a suitable composition containing vanoxerine may be prepared in the form of tablets, dragees, capsules, syrups, and aqueous or oil suspensions. The inert ingredients used in the preparation of these compositions are known in the art. For example, tablets may be prepared by mixing the active compound with an inert diluent, such as lactose or calcium phosphate, in the presence of a disintegrating agent, such as potato starch or microcrystalline cellulose, and a lubricating agent, such as magnesium stearate or talc, and then tableting the mixture by known methods.

Tablets may also be formulated in a manner known in the art so as to give a sustained release of vanoxerine. Such tablets may, if desired, be provided with enteric coatings by known method, for example by the use of cellulose acetate phthalate. Suitable binding or granulating agents are e.g. gelatine, sodium carboxymethylcellulose, methylcellulose, polyvinylpyrrolidone or starch gum. Talc, colloidal silicic acid, stearin as well as calcium and magnesium stearate or the like can be used as anti-adhesive and gliding agents.

Tablets may also be prepared by wet granulation and subsequent compression. A mixture containing vanoxerine and at least one diluent, and optionally a part of the disintegrating agent, is granulated together with an aqueous, ethanolic or aqueous-ethanolic solution of the binding agents in an appropriate equipment, then the granulate is dried. Thereafter, other preservative, surface acting, dispersing, disintegrating, gliding and anti-adhesive additives can be mixed to the dried granulate and the mixture can be compressed to tablets or capsules.

Tablets may also be prepared by the direct compression of the mixture containing the active ingredient together with the needed additives. If desired, the tablets may be transformed to dragees by using protective, flavoring and dyeing agents such as sugar, cellulose derivatives (methyl- or ethylcellulose or sodium carboxymethylcellulose), polyvinylpyrrolidone, calcium phosphate, calcium carbonate, food dyes, aromatizing agents, iron oxide pigments and the like which are commonly used in the pharmaceutical industry.

For the preparation of capsules or caplets, vanoxerine and the desired additives may be filled into a capsule, such as a hard or soft gelatin capsule. The contents of a capsule and/or caplet may also be formulated using known methods to give sustained release of the active compound.

Liquid oral dosage forms of vanoxerine may be an elixir, suspension and/or syrup, where the compound is mixed with a non-toxic suspending agent. Liquid oral dosage forms may also comprise one or more sweetening agent, flavoring agent, preservative and/or mixture thereof.

For rectal administration, a suitable composition containing vanoxerine may be prepared in the form of a suppository. In addition to the active ingredient, the suppository may contain a suppository mass commonly used in pharmaceutical practice, such as Theobroma oil, glycerinated gelatin or a high molecular weight polyethylene glycol.

For parenteral administration, a suitable composition of vanoxerine may be prepared in the form of an injectable solution or suspension. For the preparation of injectable solutions or suspensions, the active ingredient can be dissolved in aqueous or non-aqueous isotonic sterile injection solutions or suspensions, such as glycol ethers, or optionally in the presence of solubilizing agents such as polyoxyethylene sorbitan monolaurate, monooleate or monostearate. These solutions or suspension may be prepared from sterile powders or granules having one or more carriers or diluents mentioned for use in the formulations for oral administration. Parenteral administration may be through intravenous, intradermal, intramuscular or subcutaneous injections.

### EXAMPLES

The materials, methods, and examples presented herein are intended to be illustrative, and not to be construed as limiting the scope or content of the invention. Unless otherwise defined, all technical and scientific terms are intended to have their art-recognized meanings.

### Example 1

28 patients participated in a study of vanoxerine. 25 patients took a 300 mg dose of vanoxerine and 3 patients took a placebo. Each patient gave samples before administration of their dose, and then again at nine further time points, 30 minutes after administration, 1, 2, 3, 4, 6, 8, 12, and 24 hours post administration.

TABLE 1
Concentrations ng/ml

| Time | | | | | | | Total |
|---|---|---|---|---|---|---|---|
| (h) | Vanoxerine | M03 | M04 | M01 | M02 | M05 | Metabolites |
| −15 | 1.00* | 1.00 | 1.00 | 1.00 | 1.00 | 1.00 | 1.00 |
| .5 | 25.26 | 1.02 | 10.79 | 1.93 | 1.00 | 1.30 | 12.44 |
| 1 | 70.09 | 2.46 | 49.74 | 7.51 | 1.02 | 1.88 | 60.41 |
| 2 | 104.98 | 7.08 | 82.62 | 19.65 | 1.02 | 2.59 | 111.20 |
| 3 | 81.43 | 7.21 | 75.63 | 18.68 | 1.01 | 2.14 | 102.83 |
| 4 | 54.30 | 7.54 | 63.85 | 16.42 | 1.01 | 1.45 | 88.35 |
| 6 | 32.85 | 6.59 | 48.14 | 11.48 | 1.00 | 1.22 | 66.35 |
| 8 | 24.37 | 4.92 | 38.38 | 8.98 | 1.00 | 1.21 | 52.45 |
| 12 | 15.89 | 3.98 | 26.84 | 6.30 | 1.00 | 1.05 | 37.05 |
| 24 | 8.29 | 2.32 | 13.46 | 3.66 | 1.00 | 1.01 | 19.07 |

*A quantity of (1.00) represents an amount that was below the lower limit of quantitation, which is <1.139 ng/ml vanoxerine, and <1.1141 ng/ml 17-hydroxyl vanoxerine.

TABLE 2
Standard Deviations

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 43.77 | 0.12 | 15.58 | 3.20 | 0.00 | 0.80 | 19.28 |
| 1 | 61.82 | 2.51 | 49.96 | 7.08 | 0.10 | 1.13 | 59.70 |
| 2 | 100.18 | 4.70 | 51.64 | 15.31 | 0.07 | 2.56 | 70.07 |
| 3 | 80.40 | 5.40 | 49.04 | 13.63 | 0.07 | 2.31 | 64.45 |
| 4 | 55.01 | 5.32 | 39.75 | 11.31 | 0.04 | 1.16 | 52.50 |
| 6 | 35.74 | 5.10 | 31.30 | 7.90 | 0.00 | 0.87 | 41.84 |
| 8 | 30.37 | 4.05 | 25.29 | 6.74 | 0.00 | 0.94 | 33.41 |
| 12 | 24.03 | 3.15 | 17.62 | 4.70 | 0.00 | 0.27 | 23.17 |
| 24 | 10.34 | 2.11 | 8.91 | 2.76 | 0.00 | 0.03 | 12.31 |

Table 2 shows the standard deviations from the above 25 patients receiving vanoxerine. The three patients receiving a placebo are not included in the data and all data points indicated levels of vanoxerine below the lower limit of quantitation.

Tables 1 and 2, above, show tests of 25 patients with a 300 mg dose of vanoxerine. Blood was drawn from each of the test patients before the administration of the vanoxerine, and then at 9 additional time points, one half hour after administration, then 1, 2, 3, 4, 6, 8, 12, and 24 hours subsequent to administration.

The 25 patients fall into two categories: 15 fell into a category of having the majority of time point levels that were below the average mean (as identified in Table 1) “low concentration group average,” and the remaining 10 patients had the majority of time points above the average mean “high concentration group average.”

TABLE 3
Low concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 16.99 | 1.00 | 12.17 | 1.52 | 1.00 | 1.37 | 13.39 |
| 1 | 40.07 | 2.78 | 56.35 | 6.76 | 1.03 | 1.73 | 66.46 |
| 2 | 42.50 | 6.48 | 74.06 | 14.09 | 1.00 | 1.30 | 94.80 |
| 3 | 31.40 | 5.36 | 59.58 | 11.38 | 1.00 | 1.14 | 76.25 |
| 4 | 24.40 | 5.91 | 51.98 | 10.34 | 1.00 | 1.05 | 68.14 |
| 6 | 16.69 | 4.96 | 38.61 | 7.08 | 1.00 | 1.00 | 50.52 |
| 8 | 11.82 | 3.29 | 29.92 | 5.30 | 1.00 | 1.00 | 38.45 |
| 12 | 6.31 | 2.58 | 20.60 | 3.67 | 1.00 | 1.00 | 26.71 |
| 24 | 5.01 | 1.79 | 12.09 | 2.66 | 1.00 | 1.00 | 16.08 |

TABLE 4
Low concentration standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 0.00 | 0.00 | 0.00 | 0.00 | 0.00 | 0.00 | 0.00 |
| 1 | 24.47 | 0.00 | 17.68 | 1.67 | 0.00 | 0.98 | 20.45 |
| 2 | 27.50 | 3.10 | 59.32 | 7.56 | 0.13 | 1.05 | 71.04 |
| 3 | 28.16 | 4.18 | 44.96 | 9.05 | 0.00 | 0.58 | 57.77 |
| 4 | 22.66 | 3.28 | 34.95 | 7.06 | 0.00 | 0.46 | 45.53 |
| 6 | 16.11 | 3.72 | 30.77 | 7.28 | 0.00 | 0.16 | 42.04 |
| 8 | 14.20 | 3.51 | 21.42 | 3.71 | 0.00 | 0.00 | 28.30 |
| 12 | 11.19 | 2.27 | 15.60 | 2.86 | 0.00 | 0.00 | 20.34 |
| 24 | 3.07 | 1.69 | 10.44 | 1.72 | 0.00 | 0.00 | 13.40 |

TABLE 5
High concentration group average:

| Time | | | | | | | Total |
|---|---|---|---|---|---|---|---|
| (h) | Vanoxerine | M03 | M04 | M01 | M02 | M05 | Metabolites |
| −15 | 1.00 | 1.00 | 1.00 | 1.00 | 1.00 | 1.00 | 1.00 |
| .5 | 37.67 | 1.06 | 8.71 | 2.55 | 1.00 | 1.19 | 11.01 |
| 1 | 115.12 | 1.98 | 39.82 | 8.64 | 1.00 | 2.10 | 51.33 |
| 2 | 198.71 | 7.96 | 95.46 | 28.00 | 1.05 | 4.51 | 135.79 |
| 3 | 156.49 | 9.98 | 99.70 | 29.64 | 1.03 | 3.64 | 142.69 |
| 4 | 96.14 | 9.83 | 80.45 | 24.93 | 1.02 | 2.01 | 116.64 |
| 6 | 57.08 | 9.03 | 62.44 | 18.08 | 1.00 | 1.55 | 90.10 |
| 8 | 43.18 | 7.37 | 51.08 | 14.50 | 1.00 | 1.52 | 73.46 |
| 12 | 29.30 | 5.93 | 35.57 | 9.98 | 1.00 | 1.13 | 51.52 |
| 24 | 3.07 | 1.69 | 10.44 | 1.72 | 0.00 | 0.00 | 13.40 |

TABLE 6
High concentration group standard deviation:

| Time | | | | | | | Total |
|---|---|---|---|---|---|---|---|
| (h) | Vanoxerine | M03 | M04 | M01 | M02 | M05 | Metabolites |
| −15 | 0.00 | 0.00 | 0.00 | 0.00 | 0.00 | 0.00 | 0.00 |
| .5 | 62.39 | 0.19 | 12.37 | 4.71 | 0.00 | 0.45 | 18.34 |
| 1 | 72.52 | 1.19 | 31.62 | 6.52 | 0.00 | 1.26 | 38.76 |
| 2 | 96.23 | 5.50 | 60.49 | 19.21 | 0.11 | 3.17 | 82.34 |
| 3 | 77.51 | 6.85 | 58.66 | 13.99 | 0.11 | 3.12 | 70.07 |
| 4 | 63.43 | 6.50 | 46.33 | 10.60 | 0.06 | 1.67 | 54.47 |
| 6 | 44.79 | 6.26 | 38.98 | 8.02 | 0.00 | 1.35 | 48.76 |
| 8 | 40.12 | 4.97 | 32.08 | 7.21 | 0.00 | 1.48 | 38.93 |
| 12 | 33.45 | 3.74 | 22.14 | 5.13 | 0.00 | 0.42 | 26.71 |
| 24 | 14.82 | 3.02 | 11.03 | 3.24 | 0.00 | 0.05 | 14.70 |

As can be seen, in Tables 3 and 5, the low concentration group barely has plasma levels rise above 40 ng/ml at any time point in reference to vanoxerine. Whereas, the high concentration group has levels that rise to nearly 200 ng/ml at a time of two (2) hours after administration. Furthermore, the variability with regard to each of the groups is also wider. The standard deviations in Table 4 are lower than those in Table 6, (no T-test or 95% confidence was run), demonstrating that the variability was greater in the high group than the low group.

### Example 2

12 subjects received daily doses of Vanoxerine for 11 consecutive days, at doses of 25, 50, 75, and 100 mg, with a 14 day washout period between dose levels.

At 25 mg, plasma levels were not detectable after 8 hours. At 50, 75, and 100 mg doses, plasma levels were detectable at 24 hours and steady state was reached by day 8. PK was linear and dose proportional across 50, 75 and 100 mg doses. The 100 mg QD Cₘₐₓₛₛ and AUC₀₋₂₄ₛₛ suggests a trend toward non-linear PK that may become apparent at doses>100 mg QD. PK was highly variable at steady-state; Cₘₐₓ, ss, and AUC₀₋₂₄ₛₛ inter-subject variability ranged from 55-85%. The results are listed below in Table 7.

TABLE 7

| | PK Data | PK Data |
|---|---|---|
| Dose | (Mean +/− SD) C_{Max} | (Mean +/− SD) T_{1/2} |
| 50 mg | 27.5+/21.3 ng/ml | 49.39 +/− 26.18 hr |
| T_{Max} 1.27 +− 0.5 hr | | (4.71-110.57) |
| (0.5-2.0) | | |
| 75 mg | 27.4 +/− 15.5 ng/ml | 52.53 +/− 37.46 |
| | | (10.26-116.67) |
| 100 mg | 40.2 +/− 26.6 ng/ml | 15.38 +/− 43.55 |
| | | (5.56-125.00) |

Data from these studies demonstrates an increased half-life of the drug when daily doses are given. Furthermore, it was noted that heart rate and systolic blood pressure increased slightly in most subjects at 75 and 100 mg doses and did not completely return to baseline during washout between dose levels.

### Example 3

Fourteen healthy patients were given vanoxerine of 25, 75, and 125 mg, daily, for 14 days with a washout of 14 days between dose levels. A standardized meal was served 15 minutes prior to each dosing.

No significant adverse events were seen in any of the studies. Steady-state serum levels were reported within 9-11 days with disproportionately and statistically greater levels at higher doses as compared with the lower doses. The non-linear kinetics may be due to increasing bioavailability at higher doses based on a saturation of first pass metabolism. Indeed, a small proportion of the absorbed dose appears to undergo enterohepatic circulation, which may account for the considerably longer elimination half-life observed with repetitive dosing (22 hours versus 66 hours for single versus repeat administration). Steady-state plasma concentrations were achieved after 3 days with repetitive dosing, though all patients achieved steady state after 7-10 days of dosing, suggesting little potential for accumulation. This is supported by the observed lack of differential effects observed following single or repeat administration in pharmacology studies and in the absence of an effect on receptor number with repeat vanoxerine doses.

### Example 4

Four patients were given 50, 100, and 150 mg vanoxerine, daily, for 7 days.

Upon administration of 100 mg for 7 days, increases in systolic blood pressure and heart rate were seen. Similarly, during the 150 mg test, the patients also saw increases in systolic blood pressure and in heart rate. Steady-state levels were achieved within one week for all patients

### Example 5

In certain canine dosing models, adult mongrel doges, 18-23 kg, were give oral vanoxerine. Three doses were given, 90 mg at 0 min, 180 mg at 60 min, and 270 mg at 120 min. Vanoxerine plasma concentration was measured against time, and it was tested at what concentration was there an inability to reinduce atrial flutter or atrial fibrillation. All dogs studied found the inability to reintroduce AF or AFL at concentrations between 70 and 105 ng/ml.

### Example 6

3 different cohorts, each including 35 subjects were enrolled in a study with 25 taking vanoxerine and 10 receiving placebo. Cohort 1 included 200 mg vanoxerine, Cohort 2 include 200 or 300 mg of vanoxerine, and Cohort 3 included 200, 300, or 400 mg vanoxerine. The vanoxerine or identical appearing placebo was randomly assigned and administered in a double-blinded fashion.

TABLE 8
Atrial Fibrillation/Flutter history:

| | Placebo (32) | 200 mg (22) | 300 mg (25) | 400 mg (25) |
|---|---|---|---|---|
| A Flutter at | 4 (12.5) | 4 (18.2) | 4 (16) | 4 (16 |
| Entry N (%) | | | | |

Duration of Concurrent AF/AFL Episode

| | | | | |
|---|---|---|---|---|
| Mean, days | 1.84 | 2.33 | 2.43 | 1.97 |
| range, days | 0-6 | 0-6 | 0-6 | 0-7 |
| Rx same day as | 41 | 23 | 32 | 32 |
| onset, % | | | | |

Time since AF/AFL Dx

| | | | | |
|---|---|---|---|---|
| Mean, yrs | 3.9 | 4.8 | 4.5 | 5.1 |
| range, yrs | 0-21 | 0-13 | 0-13 | 1-13 |
| Rx prior DC | 44 | 45 | 52 | 32 |
| cadioversion % | | | | |

Time since last DC Cardioversion

| | | | | |
|---|---|---|---|---|
| Mean, mo | 13.6 | 15.2 | 18.2 | 21 |
| range, mo | 0-77 | 0-5 | 0-90 | 0-103 |

TABLE 9
Efficacy: Percent conversion through 4, 8, and 24 hours

| | Placebo (32) | 200 mg (22) | 300 mg (25) | 400 mg (25) |
|---|---|---|---|---|
| 0-4 hr | 13% | 18% | 40% | 52% |
| 0-8 hr | 23% | 45% | 52% | 76% |
| 0-24 hr | 38% | 59% | 64% | 84% |

Indeed, there is a significant improvement in conversion as compared to placebo at all time-points, wherein the rate of conversion or percent conversion at 0-4 hours, 0-8 hours and 0-24 hours was improved with any dose of vanoxerine. Accordingly, a measurement of the improvement comprises a comparison to the rate of conversion of placebo, wherein the improvement is based on the percent increase in conversion over placebo. The 200 mg, having an improvement of conversion of 38%, 96%, and 55% at the above time points, 300 mg: 207%, 126%, and 68%, and the 400 mg: 300%, 230%, and 121%.

TABLE 10
Time to conversion

| Log-rank test results for time conversion | P-value |
|---|---|
| Overall | 0.0005 |
| Pairwise: 200 mg versus control | 0.0838 |
| pairwise: 300 mg versus control | 0.0180 |
| pairwise: 400 mg versus control | <0.0001 |

Indeed, the time to conversion based on the P-value and the above chart provides that placebo does not have greater than a 40% conversion at any time point below 24 hours, whereas all doses of vanoxerine are greater than 40% conversion at about 7 hours, and conversion greater than 50% for all dose at 12 hours, and nearing 60% at about 16 hours.

TABLE 11
Conversion of Atrial Flutter

| | Placebo (32) | 200 mg (22) | 300 mg (25) | 400 mg (25) |
|---|---|---|---|---|
| A flutter, N | 4 | 4 | 4 | 4 |
| Conversion, % | 25% | 50% | 75% | 75% |

Definition of “pure” atrial flutter: only Atrial Flutter (no AF) seen at −30, −15, and 0 time points. Conversion at any time within 24 hours. No 1:1 AFL seen post dose in any subject.

TABLE 12
Adverse events:

| Placebo (32) | 200 mg (22) | 300 mg (25) | 400 mg (25) |
|---|---|---|---|
| 7 (22%) | 4 (18%) | 7 (28%) | 10 (40%) |
| subjects | subjects | subjects | subjects |
| reporting | reporting | reporting | reporting |
| 10 AE's | 8 AEs (1 SAE) | 12 AEs | 23 AEs (1 SAE) |

In view of doses of 200, 300 and 400 mg, there was a highly statistically significant dose dependent increase in the conversion to sinus rhythm of recent onset symptomatic AF/AFL. The highest oral dose of 400 mg achieved a conversion rate of 76% at 8 hours and 84% within 24 hours. Time to conversion curves also demonstrate increasing slope of conversion with successively higher doses, suggesting a Cₘₐₓ dependent effect.

Vanoxerine was well tolerated at all doses with only two serious adverse events, one at the 200 mg dose and one at the 400 mg dose (the 200 mg dose being an upper respiratory infection, the 400 mg dose being lower extremity edema secondary to amlodipine), neither related to the study drug. Similar to efficacy, there was a dose dependent increase in adverse events, but only the high dose event rate was notably higher than that of the placebo group. Accordingly, vanoxerine has a high degree of efficacy for the conversion of recent onset symptomatic atrial fibrillation and atrial flutter in the absence of proarrhythmia, wherein the conversion rate approaches that of DC cardioversion.

Accordingly, hemodynamic effects on heart rate and systolic blood pressure have been seen with multiple dosing of vanoxerine. Several subjects exhibited dose-related increases in heart rate and systolic blood pressure. These effects, however, do not correlate with vanoxerine concentration AUC and interpretation is further confounded by the lack of placebo-control. These effects do not immediately dissipate upon discontinuation of study drug. It is suggested that vanoxerine exerts an effect on the autonomic nervous system over the course of the study. The lack of correlation with plasma vanoxerine AUC, may be interpreted as either evidence of a significant pharmacodynamic lag in the hemodynamic effects of vanoxerine or evidence that a metabolite is responsible for the hemodynamic effects.

This study favorable compares favorably to human models where plasma concentration is charted against conversion to normal sinus rhythm. Patients that failed to convert to normal sinus rhythm had concentration of vanoxerine was between 0 ng/ml and 40 ng/ml. Conversely, patients that conversed had vanoxerine concentrations between about 30 and 130 ng/ml, with a few outliers on the low end and high end. However, most conversions occurred in the range of about 60 ng/ml. Accordingly, modifying doses to reach 60 ng/ml or higher is preferred for effective conversion to normal sinus rhythm.

Accordingly, in view of the data, certain methods of diet modification may be suitable for normalizing or minimizing the variability with regard to a single dosage of vanoxerine or one or more of the metabolites identified herein. Modulation of diet and or of a dose of vanoxerine provides for greater accuracy with regard to target plasma concentrations for the treatment of cardiac arrhythmia. Utilization of food intake concurrently with vanoxerine allows for appropriate modulation of Cₘₐₓ and tₘₐₓ and AUC such that variability is minimized with patients. Therefore, the methods provided for herein, provide for greater accuracy with regard to target physiological levels (blood, plasma, and other tissues), thus increasing the safety profile, improving efficacy of treatment, and minimizing side effects that may be associated with treatment.

Although the present invention has been described in considerable detail, those skilled in the art will appreciate that numerous changes and modifications may be made to the embodiments and preferred embodiments of the invention and that such changes and modifications may be made without departing from the spirit of the invention. It is therefore intended that the appended claims cover all equivalent variations as fall within the scope of the invention.

What is claimed is:

1. A method for administering vanoxerine for treatment of cardiac arrhythmia comprising:
a. administering a first dose of vanoxerine to a patient;
b. determining the bioavailability of the patient by measuring the physiological concentration of vanoxerine;
c. calculating an effective dose of vanoxerine to be administered with a meal of a pre-determined fat content to be taken concurrently to modify the physiological concentration; and
d. administering the effective dose of vanoxerine with the pre-determined meal.

2. The method of claim 1 wherein the meal has a fat content of at least 70 g and is taken directly before or concurrently with the vanoxerine dose.

3. The method of claim 1 wherein the meal has a fat content of at least 50 g and is taken directly before or concurrently with the vanoxerine dose.

4. The method of claim 1 wherein the meal has a fat content of at least 20 g and is taken directly before or concurrently with the vanoxerine dose.

5. The method of claim 1 wherein the meal has a fat content of less than 10 g and is taken directly before or concurrently with the vanoxerine dose.

6. The method of claim 1 wherein the meal is omitted and fasting is instituted with the effective dose of vanoxerine.

7. A method for achieving a pre-determined plasma level comprising:
a. administering a first dose of vanoxerine concurrently with a high-fat meal;
b. measuring the physiological concentration of vanoxerine;
c. comparing the physiological concentration to the pre-determined physiological concentration;
d. modifying a further dose of vanoxerine to be given concurrently with a high-fat meal; and
e. administering the second dosage of vanoxerine in conjunction with the high-fat meal.

8. The method of claim 7 wherein the pre-determined physiological concentration is taken two hours post administration.

9. The method of claim 7 wherein the pre-determined physiological concentration is taken four hours post administration.

10. The method of claim 7 wherein said high fat food comprises at least 20 g of fat.

11. The method of claim 7 wherein said high fat food comprises at least 50 g of fat.

12. The method of claim 7 wherein said high fat food comprises at least 70 g of fat.

13. The method of claim 7 wherein said pre-determined physiological concentration is measured from the blood plasma.

14. The method of claim 13 wherein the blood plasma concentration is greater than 60 ng/ml.

15. A method of minimizing variability of physiological concentrations for treatment of cardiac arrhythmia with vanoxerine comprising:
a. determining a target physiological concentration;
b. administering a first dose of a drug comprising vanoxerine to a patient;
c. measuring the physiological concentration of vanoxerine in said patient; and
d. instructing patient to consume a high-fat meal concurrently with a further dose of vanoxerine.

16. The method of claim 14 wherein said high-fat meal comprises at least 20 g of fat.

17. The method of claim 14 wherein said high-fat meal comprises at least 50 g of fat.

18. The method of claim 14 wherein said high-fat meal comprises at least 70 g of fat.

19. The method of claim 14 further comprising the step of modifying the further dose of vanoxerine.

20. A method of administration of vanoxerine to a patient comprising administering a first dose of vanoxerine to a patient under fasting conditions and administering a second dose of vanoxerine to said same patient about 1-2 hours after said first administration, wherein said second dose is taken concurrently with a high-fat meal.

21. The method of claim 19, wherein said first dose of vanoxerine is between 200 and 400 mg.

22. The method of claim 19, wherein said high-fat meal comprises at least 20 g of fat.

23. The method of claim 19, wherein said high-fat meal comprises at least 50 g of fat.

24. The method of claim 19, wherein said high-fat meal comprises at least 70 g of fat.
